(19) United States
(12) Patent Application Publication
(10) Pub. No.: US 2025/0331775 A1
(43) Pub. Date: Oct. 30, 2025

(54) FULLY IMPLANTABLE AND DETACHABLE HIGH CHANNEL NEURAL INTERFACE DEVICE, METHOD FOR PROCEEDING ELECTRODE AND METHOD FOR REPLACING DEVICE

(71) Applicant: BCIFLEX MEDICAL TECHNOLOGY CO., LTD., Beijing (CN)

(72) Inventors: Bo ZENG, Beijing (CN); Qi SONG, Beijing (CN); Jinfen WANG, Beijing (CN); Huihui TIAN, Beijing (CN); Guiqiang YANG, Beijing (CN)

(21) Appl. No.: 19/188,128

(22) Filed: Apr. 24, 2025

(30) Foreign Application Priority Data
Apr. 30, 2024 (CN) 202410534185.8

(51) Int. Cl. A61B 5/00; A61B 5/293; A61B 5/294; A61N 1/375

(52) U.S. Cl. CPC A61B 5/686; A61B 5/293; A61B 5/294; A61B 2560/0443; A61B 2562/125; A61B 2562/164; A61B 2562/227; A61N 1/3754

(57) ABSTRACT

A neural interface device, a method for producing an electrode and a method for replacing a device are disclosed. The device includes an implantable case including a bottom shell and a press cover sealed with each other, a feed-through plate, a neural electrode and an interposer connector. The case accommodates a neural signal circuit. First conductive contacts of the plate is connected to the circuit. A distal electrode site portion of the electrode is electrically connected to a proximal contact portion. The proximal portion and the connector are sealed between the cover and the shell. An interconnect portion and the distal portion protrude out between the shell and the cover. The proximal portion is electrically connected to the conductive contact of the plate via the connector. The case is individually replaced, the electrode is kept in a tissue, detachability and sealing performance are balanced and operation risk is reduced.

### CROSS-REFERENCE TO RELATED APPLICATION

The present application claims priority to Chinese Patent Application No. 202410534185.8 entitled “FULLY IMPLANTABLE AND DETACHABLE HIGH CHANNEL NEURAL INTERFACE DEVICE, METHOD FOR PROCEEDING ELECTRODE AND METHOD FOR REPLACING DEVICE” and filed with the CNIPA on Apr. 30, 2024, the content of which is incorporated herein by reference in its entirety.

### FIELD

The present disclosure relates to the technical field of medical devices, and more particularly, to a fully implantable and detachable high channel neural interface device.

### BACKGROUND

Regarding a fully implantable and detachable high channel neural interface device in the related art, especially a fully implantable high-flux neural implanted device, a device body and its high-flux electrode need to be implanted into the human body together. However, the device body and the high-flux electrode are formed as an integrated structure and cannot be detachable from each other. Therefore, when the battery is fully depleted or the device fails or needs to be replaced, the device body and the high-flux electrode need to be taken out together and then a new device needs to be implanted. There is a great danger in the process of electrode implantation, because the difficulty in operation is rather high and thus a great damage to the human body may be generated. In addition, in the fully implantable and detachable high channel neural interface device, an internal circuit needs to be isolated from an external electrode, but it is difficult to separate the electrode from the device.

### SUMMARY

The present disclosure is intended to solve at least one of the technical problems existing in the prior art. One aim of the present disclosure is to provide a fully implantable and detachable high channel neural interface device. The high channel implantable device can keep electrode in human body, balance detachability and sealing performance, reduce risk of operation, and reduce the damage to human body.

Another aim of the present disclosure is to provide a method for producing a flexible neural electrode.

Yet another aim of the present disclosure is to provide a method for replacing the fully implantable and detachable high channel neural interface device.

To achieve the above aims, an embodiment in a first aspect of the present disclosure provides a fully implantable and detachable high channel neural interface device. The fully implantable and detachable high channel neural interface device includes an implantable case, a first feed-through plate, a flexible neural electrode, and an interposer connector.

The implantable case includes a bottom shell and a press cover. The press cover is removably mounted on the bottom shell and is sealed with the bottom shell. The implantable case is configured with a neural signal circuit therein.

The first feed-through plate is arranged on the bottom shell. The first feed-through plate has a first surface facing an interior of the implantable case and a second surface facing away from the first surface. The first feed-through plate is provided with a plurality of first conductive contacts extending from the first surface to the second surface. The plurality of first conductive contacts are electrically connected to a neural signal circuit inside the implantable case at the first surface.

The flexible neural electrode includes a proximal contact portion, an interconnect portion and a distal electrode site portion. The distal electrode site portion includes a plurality of electrode sites and is electrically connected to the proximal contact portion by the interconnect portion. The proximal contact portion of the flexible neural electrode and the interposer connector being sealed between the press cover and the bottom shell. The interconnect portion and the distal electrode site portion of the flexible neural electrode protrude out from between the bottom shell and the press cover. The proximal contact portion is a planar structure and is detachably connected to the bottom shell; and

The interposer connector is arranged between the proximal contact portion of the flexible neural electrode and the second surface of the first feed-through plate. The proximal contact portion of the flexible neural electrode is electrically connected to the first conductive contact of the first feed-through plate via the interposer connector so as to allow the distal electrode site portion of the flexible neural electrode to be electrically connected to the neural signal circuit in the implantable case.

The fully implantable and detachable high channel neural interface device According to the embodiment of the present disclosure can keep the electrode in human body, balance detachability and sealing performance, reduce risk of operation, and reduce the damage to human body.

According to some specific embodiments of the present disclosure, the interposer connector is configured in such a way that when a pressure applied by the press cover to the proximal contact portion of the flexible neural electrode reaches a first threshold, the proximal contact portion of the flexible neural electrode is electrically connected to the first conductive contact of the first feed-through plate via the interposer connector so as to allow the distal electrode site portion of the flexible neural electrode to be electrically connected to the neural signal circuit in the implantable case.

According to some specific embodiments of the present disclosure, the neural signal circuit includes a neural signal acquisition circuit and/or a neural signal stimulation circuit.

Furthermore, the press cover is secured with the bottom shell of the implantable case via screws or mechanical interlocks.

According to some specific embodiments of the present disclosure, one side of the bottom shell is configured with a recess portion, the first feed-through plate, the interposer connector, and the proximal contact portion of the flexible neural electrode are all mounted on the recess portion, the press cover seals the first feed-through sheet, the interposer connector, and the proximal contact portion of the flexible neural electrode to the recess portion.

According to some specific embodiments of the present disclosure, a sealing member around the first feed-through plate is provided between the bottom shell and the press cover, the distal electrode site portion of the flexible neural electrode protrudes out of the implantable case by protruding out from between the sealing member and the press cover.

According to some specific embodiments of the present disclosure, the bottom shell is configured with an alignment mechanism for positioning and mounting with the press cover, the interposer connector and the proximal contact portion of the flexible neural electrode.

Furthermore, the alignment mechanism includes at least one alignment pin arranged on the bottom shell. The press cover, the interposer connector and the proximal contact portion of the flexible neural electrode are all configured with an alignment slot hole corresponding to a position of the alignment pin. The alignment pin passes through the alignment slot hole.

According to some specific embodiments of the present disclosure, the alignment mechanism further includes an alignment boss being arranged on the bottom shell and protruding towards a direction of the press cover. A bottom of the press cover is configured with a limiting groove that fits with the alignment boss.

According to some specific embodiments of the present disclosure, a bottom surface of the press cover is configured with a pressing plate corresponding to a position of the first feed-through plate. The pressing plate compresses the proximal contact portion of the flexible neural electrode against the interposer connector in a value reaching a first threshold, to allow the proximal contact portion of the flexible neural electrode and the first conductive contact of the first feed-through plate of the implantable case are electrically connected to each other through the interposer connector.

According to some specific embodiments of the present disclosure, the first feed-through plate includes a first insulating layer, the first conductive contact passes through the first insulating layer along a thickness direction.

Furthermore, the plurality of first conductive contacts are arranged on the first feed-through plate in an array and the number of the first conductive contacts is not less than 100.

Furthermore, the first insulating layer is made of ceramic or glass.

According to some specific embodiments of the present disclosure, the proximal contact portion of the flexible neural electrode has a shape of polygon or circle, the interposer connector has a shape matching the shape of the proximal contact portion of the flexible neural electrode.

According to some specific embodiments of the present disclosure, the fully implantable and detachable high channel neural interface device further includes a second feed-through plate. The second feed-through plate is electrically connected to the proximal contact portion of the flexible neural electrode. The second feed-through plate is provided with a plurality of second conductive contacts passing through the second feed-through plate along a thickness direction of the second feed-through plate, and is electrically connected to the distal electrode site portion of the flexible neural electrode via the interconnect portion of the flexible neural electrode.

Furthermore, the second feed-through plate includes a second insulating layer and plurality second conductive contacts which passes through the second insulating layer along the thickness direction.

According to some specific embodiments of the present disclosure, the second insulating layer is made of ceramic or glass.

According to some specific embodiments of the present disclosure, the interposer connector includes an insulating medium and a plurality of conductive connectors arranged on the insulating medium, and the proximal contact portion of the flexible neural electrode is electrically connected with the first conductive contact of the first feed-through plate via the conductive connector.

According to some specific embodiments of the present disclosure, the flexible neural electrode is configured as a stacked structure and includes a first flexible insulating layer, a second flexible insulating layer and a first conductive layer. The first conductive layer is positioned between the first flexible insulating layer and the second flexible insulating layer. The first conductive layer includes a plurality of electrodes. The plurality of electrodes each includes a contact structure arranged at the proximal contact portion, an interconnecting wire arranged at the interconnect portion, and an electrode site structure arranged at the distal electrode site portion. The contact structure and the electrode site structure are exposed to at least one of the first flexible insulating layer and the second flexible insulating layer.

Furthermore, the flexible neural electrode further includes: a third flexible insulating layer and a second conductive layer. The second conductive layer is positioned between the second flexible insulating layer and the third flexible insulating layer. The second conductive layer includes a plurality of electrodes. The plurality of electrodes each includes a contact structure arranged at the proximal contact portion, an interconnecting wire arranged at the interconnect portion, and an electrode site structure arranged at the distal electrode site portion.

The contact structure and the electrode site structure are exposed to at least one of the third flexible insulating layer and the second flexible insulating layer/the first flexible insulating layer.

According to some specific embodiments of the present disclosure, the flexible neural electrode includes: at least one of an epidural electroencephalogram electrode, a subdural electroencephalogram electrode, an intracortical electrode, and a depth electrode.

According to some specific embodiments of the present disclosure, the fully implantable and detachable high channel neural interface device further includes a data processing module configured to process a neural signal collected from the distal electrode site portion of the flexible neural electrode and/or generate a stimulation signal acting on the distal electrode site portion of the flexible neural electrode.

According to some specific embodiments of the present disclosure, the fully implantable and detachable high channel neural interface device further includes a protection module configured to perform shutoff protection in case of malfunction or abnormal situation of the fully implantable and detachable high channel neural interface device.

According to some specific embodiments of the present disclosure, the implantable case further includes a battery module configured to provide electric power to the fully implantable and detachable high channel neural interface device.

Furthermore, the battery module is a rechargeable battery.

According to some specific embodiments of the present disclosure, the fully implantable and detachable high channel neural interface device further includes an external controller. The external controller is equipped with a wireless communication module for wireless communication with the fully implantable and detachable high channel neural interface device.

A method for producing a flexible neural electrode is provided according to an embodiment in a second aspect of the present disclosure. The method is applied in the fully implantable and detachable high channel neural interface device according to any of the above embodiment of the present disclosure. The method includes the following steps S1 to S6.

At S1, a sacrificial layer is formed on a support by photolithography and metal coating.

At S2, a first flexible insulating layer is formed on the sacrificial layer by spin coating.

At S3, a first conductive layer is formed on the first flexible insulating layer by photolithography and metal deposition.

At S4, a second flexible insulating layer is formed by spin coating on the first conductive layer and the first flexible insulating layer that have been formed.

At S5, an overall structure of the flexible neural electrode is formed by etching the first flexible insulating layer and the second flexible insulating layer, and a contact structure of the proximal contact portion and an electrode site structure of the distal electrode site portion are exposed by etching the second flexible insulating layer.

At S6, the flexible neural electrode is placed in a sacrificial layer removal solution to partially or completely detach the flexible neural electrode from the substrate, and the detached support is subsequently removed so as to obtain the freestanding flexible neural electrode.

The method for producing the flexible neural electrode of the embodiment of the present disclosure, when applied to the fully implantable and detachable high channel neural interface device in any of the embodiments of the present disclosure, can keep the electric electrode in human body, balance detachability and sealing performance, reduce the risk of operation and reduce the damage to the human body.

A method for replacing a fully implantable and detachable high channel neural interface device is provided according to the third aspect embodiment of the present disclosure.

The method is applied to the fully implantable and detachable high channel neural interface device according to the above embodiment of the present disclosure. The method includes:
taking an original implantable case out;
implanting a new implantable case into a target tissue; and
electrically connecting the proximal contact portion of an original flexible neural electrode to the first feed-through plate of the new implantable case.

The method for replacing the fully implantable and detachable high channel neural interface device according to the embodiment of the present disclosure, when applied to the fully implantable and detachable high channel neural interface device in any of the above embodiments, can keep the electrode in human body, balance detachability and sealing performance, reduce the risk of operation is small and reduce the damage to the human body.

Furthermore, when the original implantable case is taken out, the original flexible neural electrode is not taken out.

Furthermore, the proximal contact portion of the original flexible neural electrode is connected to the first feed-through plate of the new implantable case via a detachable connection manner.

Additional aspects and advantages of the present disclosure are partly given in the description below, and some will become apparent from the description below or become known through the practice of the present disclosure.

### BRIEF DESCRIPTION OF THE DRAWINGS

The above and/or additional aspects and advantages of the present disclosure will become apparent and readily comprehensible from the description of the embodiments in conjunction with the following drawings.

FIG. 1 is a schematic structure diagram of a fully implantable and detachable high channel neural interface device according to an embodiment of the present disclosure.

FIG. 2 is an explosive view of a fully implantable and detachable high channel neural interface device according to an embodiment of the present disclosure.

FIG. 3 is a section view showing a part of a fully implantable and detachable high channel neural interface device according to an embodiment of the present disclosure.

FIG. 4 is a schematic diagram of a press cover of a fully implantable and detachable high channel neural interface device according to an embodiment of the present disclosure.

FIG. 5 is a schematic diagram showing an interior of a fully implantable and detachable high channel neural interface device according to an embodiment of the present disclosure.

FIG. 6 is a schematic diagram showing an interior of a fully implantable and detachable high channel neural interface device according to another embodiment of the present disclosure.

FIG. 7 is a schematic diagram showing an interior of a fully implantable and detachable high channel neural interface device according to yet another embodiment of the present disclosure.

FIG. 8 is a schematic diagram of a flexible neural electrode having a second feed-through plate in a fully implantable and detachable high channel neural interface device according to an embodiment of the present disclosure.

FIG. 9 is a structure diagram of a flexible neural electrode of a fully implantable and detachable high channel neural interface device according to an embodiment of the present disclosure.

### REFERENCE NUMERALS

fully implantable and detachable high channel neural interface device 1, implantable case 100, bottom shell 110, press cover 120,
first feed-through plate 200, first conductive contact 210, flexible neural electrode 300, proximal contact portion 310,
interconnect portion 320, distal electrode site portion 330,
interposer connector 400, second feed-through plate 500, first flexible insulating layer 11, second flexible insulating layer 12,
first conductive layer 13, third flexible insulating layer 14, second conductive layer 15,
recess portion 111, sealing member 600, alignment boss 112, alignment pin 113, threaded hole 114, limiting groove 121,
alignment slot hole 122, pressing plate 123.

### DETAILED DESCRIPTION

In the description of the present disclosure, it is to be understood that, the terms such as “center”, “longitudinal”, “transverse”, “length”, “width”, “thickness” and “up”, “down”, “front”, “rear”, “left”, “right”, “vertical”, “horizontal”, “top”, “bottom”, “inside” and “outside”, “axial”, “radial” and “circumferential” and so on indicate orientation or positional relations that are on the basis of the orientational or positional relations illustrated in the drawings. Each of those terms is merely for the convenience and simplification of the description of the present disclosure, does not indicate or imply that the indicated device or component must be in a particular orientation or must be constructed and operated in a particular orientation, and therefore cannot be construed as limiting the present disclosure.

In the description of the present disclosure, “first feature” and “second feature” may include one or more of the features.

In the description of the present disclosure, “a plurality of” means two or more than two.

In the description of the present disclosure, a first feature being “above” or “below” a second feature may include direct contact between the first and second features, or indirect connection of the first feature with second feature through an additional feature between them.

In the description of the present disclosure, a first feature being “above” and “over” a second feature includes the first feature being directly above the second feature and the first feature being diagonally above the second feature, or simply indicates that the first feature is at a higher level than the second feature.

A fully implantable and detachable high channel neural interface device 1 according to an embodiment of the present disclosure will be described below with reference to the attached drawings.

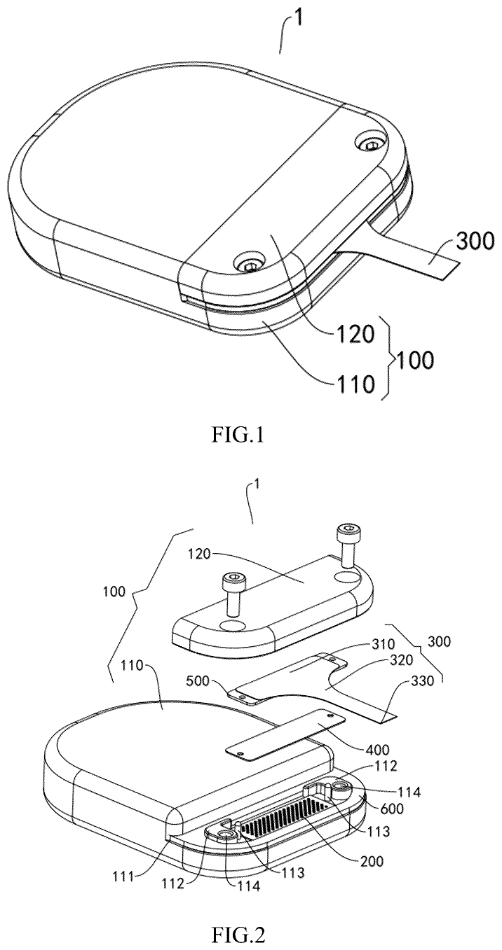

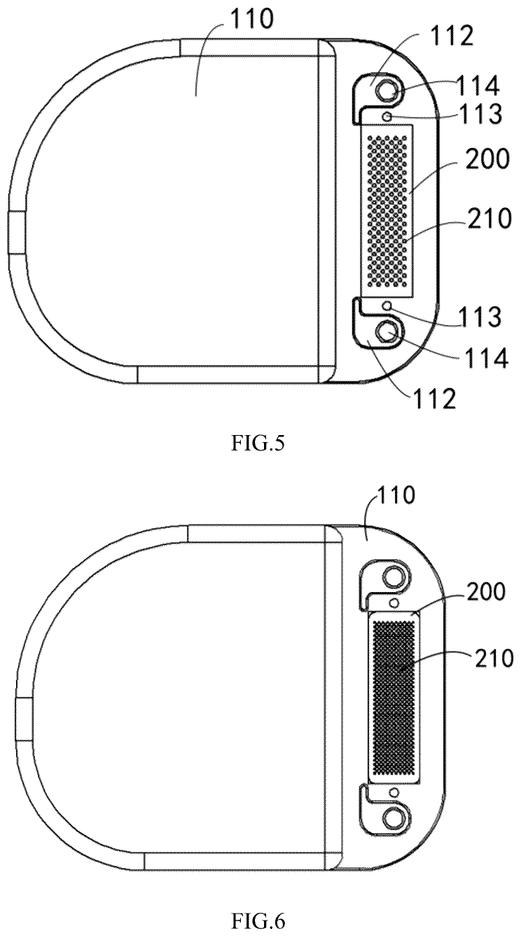

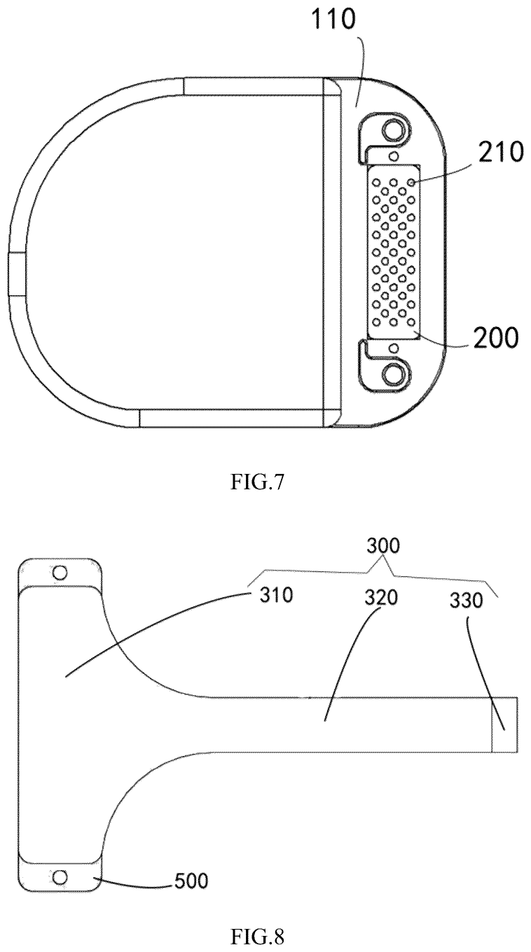

As is shown in FIG. 1 to FIG. 9, the fully implantable and detachable high channel neural interface device 1 (hereinafter referred to as device 1) according to an embodiment of the present disclosure includes an implantable case 100, a first feed-through plate 200, a flexible neural electrode 300 and an interposer connector 400.

The implantable case 100 includes a bottom shell 110 and a press cover 120. The press cover 120 is removably mounted on the bottom shell 110 and is sealed with the bottom shell 110. The implantable case 100 is configured with a neural signal circuit therein.

The first feed-through plate 200 is arranged on the bottom shell 110. The first feed-through plate 200 has a first surface facing an interior of the implantable case 100 and a second surface facing away from the first surface. The first feed-through plate 200 is provided with a plurality of first conductive contacts 210 extending from the first surface to the second surface. The plurality of first conductive contacts 210 are electrically connected to a neural signal circuit inside the implantable case 100 at the first surface.

The flexible neural electrode 300 includes a proximal contact portion 310, an interconnect portion 320 and a distal electrode site portion 330. The distal electrode site portion 330 includes a plurality of electrode sites and is electrically connected to the proximal contact portion 310 by the interconnect portion 320. The proximal contact portion 310 of the flexible neural electrode 300 and the interposer connector 400 are sealed between the press cover 120 and the bottom shell 110. The interconnect portion 320 and the distal electrode site portion 330 of the flexible neural electrode 300 protrude out from between the bottom shell 110 and the press cover 120. The proximal contact portion 310 is a planar structure and is detachably connected to the bottom shell 110.

The interposer connector 400 is arranged between the proximal contact portion 310 of the flexible neural electrode 300 and the second surface of the first feed-through plate 200. The proximal contact portion 310 of the flexible neural electrode 300 is electrically connected to the conductive contacts of the first feed-through plate 200 via the interposer connector 400 so as to allow the distal electrode site portion 330 of the flexible neural electrode 300 to be electrically connected to the neural signal circuit in the implantable case 100.

For example, the flexible neural electrode 300 is specifically a high-flux neural electrode. The flexible neural electrode 300 is a flexible neural electrode 300 to be implanted in the brain, brain subdural part, or spinal cord. The interposer connector 400 includes a substrate and a conductive contact array passing through the substrate. For example, the interposer connector 400 can be an Anisotropic Conductive Film (ACF), a Land Grid Array (LGA) connector, or other two-dimensional lattice connectors with first and second surfaces. The interposer connector 400 is applied for electric interconnection between the proximal contact portion 310 of flexible neural electrode 300 of high-flux nerve and the electronic device 1. The bottom shell 110 and press cover 120 are mounted together through screws. The first feed-through plate 200 has a long-rectangular shape. The proximal contact portion 310 is located in the implantable case 100 and is connected to the first feed-through plate 200. The width of the interconnect portion 320 gradually decreases from the proximal contact portion 310 to the distal electrode site portion 330. The distal electrode site portion 330 extends beyond the implantable case 100. The implantable case 100 has a titanium alloy structure, which is of light weight, high strength and excellent biocompatibility and excellent corrosion resistance.

According to the device 1 of the embodiment of the present disclosure, a detachable electrical interconnection is achieved by setting the interposer connector 400 and the flexible neural electrode 300. In this way, the part of device 1 other than the flexible neural electrode 300 is configured as a detachable structure, and thus it can only keep the flexible neural electrode 300 in human body, thereby avoiding damage to human brain caused by disassembly of the flexible neural electrode 300. In addition, a surface contact is formed between the first feed-through plate 200 and the flexible neural electrode 300. The provision of the press cover 120 can allow the flexible neural electrode 300 to press against the first conductive contact 210 of the first feed-through plate 200 so as to ensure a reliable electrical connection of the flexible neural electrode 300. In addition, the press cover 120 and the bottom shell 110 form a seal connection, so that the flexible neural electrode 300 and the first feed-through plate 200 inside the implantable case 100 are isolated from the external environment, thereby balancing detachability and sealing performance. When the device 1 needs to be replaced or disassembled, it is only needed to separate the press cover 120 from the bottom shell 110 and take the bottom shell 110 and the press cover 120 out and keep the flexible neural electrode 300 in the human body. Therefore, for the device according to the embodiment of the present disclosure, there is no need to operate on the electrode, thus canceling operations of taking out and re-implanting the flexible neural electrode 300, greatly reducing the surgery time duration, reducing the risk of the surgery and the patient's surgical pain.

The proximal contact portion 310 is located in the implantable case 100. The proximal contact portion 310 has a shape matching the shape of the interposer connector 400. The interconnect portion 320 is connected to a side of the proximal contact portion 310 that is facing an exterior of the implantable case 100. The interconnect portion 320 protrudes out of the implantable case 100 by protruding from between the bottom shell 110 and the press cover 120 and is implanted into the brain.

For example, the proximal contact portion 310, the interposer connector 400 and the first feed-through plate 200 all have a long rectangular shape. The interconnect portion 320 is connected to a side of the proximal contact portion 310 in the width direction of the proximal contact portion 310. The interconnect portion 320 is connected to the middle of the proximal contact portion 310 in a length direction of the proximal contact portion 310. The extension directions of the proximal contact portion 310 and the interconnect portion 320 are perpendicular to each other, and the proximal contact portion 310 and the interconnect portion 320 are connected via a cambered transition. The press cover 120 and bottom shell 110 are sealed with two opposite sides of interconnect portion 320 respectively. Since the proximal contact portion 310 has a shape matching the shape of the interposer connector 400 and the shape of the first feed-through plate 200, the proximal contact portion 310 and the first feed-through plate 200 are electrically connected to the opposite surfaces of the interposer connector 400 correspondingly. The proximal contact portion 310, the interposer connector 400 and the first feed-through plate 200 are laminated and press against the bottom shell 110 together by the press cover 120. A reliable electrical interconnection between the flexible neural electrode 300 and the first feed-through plate 200 can be achieved by deformation of the conductive contacts of the interposer connector 400. In addition, the interposer connector 400 is used to ensure the accuracy of the connection between the flexible neural electrode 300 and the first feed-through plate 200.

Therefore, according to the device of the embodiment of the present disclosure, the electrode can be kept in the human body, detachability and sealing performance are balanced, the risk of operation is small and the damage to human body is small.

In some specific embodiments of the present disclosure, the interposer connector 400 is configured in such a way that when a pressure applied by the press cover 120 to the proximal contact portion 310 of the flexible neural electrode 300 reaches a first threshold, the proximal contact portion 310 of the flexible neural electrode 300 is electrically connected to the conductive contact of the first feed-through plate 200 via the interposer connector 400 so as to allow the distal electrode site portion 330 of the flexible neural electrode 300 to be electrically connected to the neural signal circuit in the implantable case 100.

Through applying a pressure to the proximal contact portion 310 by the press cover 120, the proximal contact portion 310 of the flexible neural electrode 300 is electrically connected to the first conductive contact 210 of the first feed-through plate 200 when the pressure reaches the first threshold. The structure of the press cover 120 and the bottom shell 110 of the implantable case 100 ensures a stable electrical connection. For example, the first threshold is 1.5 kgf/cm². The device 1 uses a sensor to collect the torque of the proximal contact portion 310 of the flexible neural electrode 300 in a certain area, so that the device 1 is switched on when the torque of the compression area reaches the first threshold, thereby achieving the electric interconnection of first feed-through plate 200, the interposer connector 400 and the neural signal circuit.

In some specific embodiments of the present disclosure, the neural signal circuit includes a neural signal acquisition circuit and/or a neural signal stimulation circuit.

By setting the neural signal acquisition circuit in the shell, the neural state of the human brain can be recognized and the health state can be monitored in real time. By setting the neural signal stimulation circuit in the shell, the high frequency electrical stimulation can be released, which acts as an electric brain stimulation. The electricity stimulates the growth and differentiation of nerve cells, promotes nerve regeneration and repair, and accelerates the repair process of nerves.

Furthermore, the press cover 120 is secured with the bottom shell 110 of the implantable case 100 via screws or mechanical interlocks, thus the press cover 120 and the bottom shell 110 are removable. When the device 1 needs to be replaced or removed, it only needs to separate the press cover 120 from bottom shell 110 and take the bottom shell 110 and the press cover 120 out. The flexible neural electrode 300 is just kept in the human body.

In some specific embodiments of the present disclosure, as shown in FIG. 2, one side of the bottom shell 110 is configured with a recess portion 111 having different heights. The first feed-through plate 200, the interposer connector 400, and the proximal contact portion 310 of the flexible neural electrode 300 are all mounted on the recess portion 111. The press cover 120 is mounted on recess portion 111 by a fastener, and seals the first feed-through plate 200, the interposer connector 400, and the proximal contact portion 310 of the flexible neural electrode 300 to the recess portion.

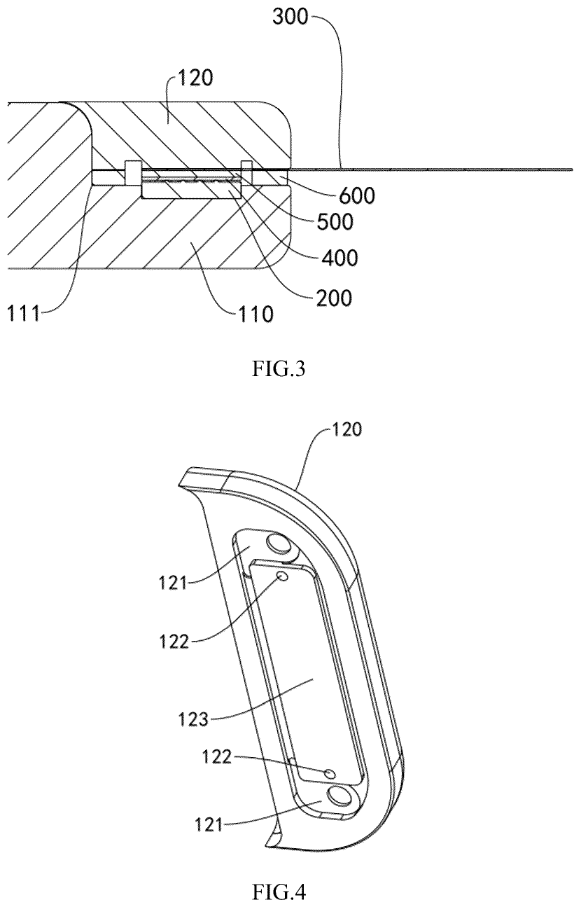

Furthermore, as shown in FIG. 2 and FIG. 3, the recess portion 111 has a top step surface, a bottom step surface and a side step surface. The press cover 120 is attached to the side step surface. A side surface of the press cover 120 is flush with a side surface of the bottom shell 110 and a top surface of the press cover 120 is flush with the top step surface.

Therefore, the press cover 120 and the bottom shell 110 can form the overall external outline, which has a high integrated structure. When the device 1 is implanted into the human skull, the shape structure is integrated and the edges are smooth, thus the damage to the human body is small.

The recess portion 111 on which the flexible neural electrode 300, the interposer connector 400 and the first feed-through plate 200 are mounted is formed at one side of the bottom shell 110, and the recess portion 111 is located the side from which the flexible neural electrode 300 protrudes. Therefore, the bottom shell 110 only needs to be sealed with the press cover 120 at the one side thereof, a fully sealed structure can be formed at the other side of the bottom shell 110, and a closed acquisition circuit, an electric stimulation circuit and other structures can be arranged in the bottom shell 110, thereby achieving the functions of acquisition and electric stimulation. Therefore, it can effectively ensure the sealing of structures such as the flexible neural electrode 300, the interposer connector 400, the first feed-through plate 200 installed within the implantable case 100.

In some specific embodiments of the present disclosure, as shown in FIG. 2 and FIG. 3, a sealing member 600 around the first feed-through plate 200 is provided between the bottom shell 110 and the press cover 120. The distal electrode site portion 330 of the flexible neural electrode 300 protrudes out of the implantable case 100 by protruding out from between the sealing member 600 and the press cover 120.

For example, the sealing member 600 is attached to the side step surface of the recess portion 111 and is attached to an overall external outline of the bottom shell 110. The seal fills the gap between the press cover 120 and the bottom shell 110. Thus, a space isolated from the outside is formed inside the implantable case 100. The proximal contact portion 310 of the flexible neural electrode 300, the first feed-through plate 200 and the interposer connector 400 are sealed in the space, so as to ensure the sealing performance of the device 1. The sealing member 600 can be adapted to the pressure of the press cover 120 and generate a certain deformation, so that the press cover 120 can apply a certain pressure on the flexible neural electrode 300, the interposer connector 400 and the first feed-through plate 200, which plays a sealing role while ensuring the electrical connection, thereby making sure that no fluids get into the implantable case 100.

In some specific embodiments of the present disclosure, as shown in FIG. 2, the bottom shell 110 is configured with an alignment mechanism for positioning and mounting with the press cover 120, the interposer connector 400 and the flexible neural electrode 300.

Furthermore, as shown in FIG. 2 and FIG. 5, the alignment mechanism includes at least one alignment pin 113. The alignment pin 113 is arranged on the bottom shell 110. The press cover 120, the interposer connector 400 and the proximal contact portion 310 of the flexible neural electrode 300 are all configured with an alignment slot hole 122 corresponding to a position of the alignment pin 113. The alignment pin 113 passes through the alignment slot hole 122.

For example, there are a plurality of alignment pins 113. The first feed-through plate 200 is located among the plurality of alignment pins 113 in a length direction of the first feed-through plate 200. The alignment pins 113 provide alignment for the press cover 120, the interposer connector 400, and the proximal contact portion 310 of the flexible neural electrode 300 through corresponding alignment slot holes. Therefore, a detachable function can be realized, so a to ensure the accuracy of the installation positions of the proximal contact portion 310 and the interposer connector 400. In addition, the interposer connector 400 and the proximal contact portion 310 can be matched to the position of the press cover 120, and the pressure applied by the press cover 120 is more uniform.

In some specific embodiments of the present disclosure, as shown in FIG. 2, the alignment mechanism further includes an alignment boss 112. The alignment boss 112 is arranged on the bottom shell 110 and protrudes towards a direction of the press cover 120. The alignment boss 112 is located at each of two sides of the first feed-through plate 200. A bottom of the press cover 120 is configured with a limiting groove 121 that fits with the alignment boss 112.

The first feed-through plate 200 is arranged on the bottom of the bottom shell 110. The alignment boss 112 is provided with a threaded hole 114. In the bottom shell 110, a threaded fastener can be provided to pass through corresponding positions of the alignment boss 112 and the limiting groove 121 so as to connect with the threaded hole 114 of the alignment boss 112.

The alignment pin 113 is arranged to be adjacent to the alignment boss 112. The alignment slot hole 122 is arranged to be adjacent to the limiting groove 121. The alignment pin 113 and the alignment boss 112 are all located at each of two sides of the first feed-through plate 200. The alignment slot hole 122 and the limiting groove 121 are provided to be corresponding to the alignment pin 113 and the alignment boss 112 respectively. The alignment pin 113 is installed in the alignment slot hole 122, so that the positions of the press cover 120 and the bottom shell 110 can be predetermined, making a subsequent connection with threaded fasteners more stable.

The alignment boss 112 is correspondingly fitted and installed into the limiting groove 121, which ensures the accuracy of the connection positions of the press cover 120 and the bottom shell 110, so as to ensure that the press cover 120 can compress tightly the flexible neural electrode 300 at the exact position and ensure the reliability of the electrical connection. The threaded fastener passes through the through hole of the limiting groove 121 to be connected to the threaded hole 114 of the alignment boss 112, so that a greater compression force can be provided by the press cover 120 and the bottom shell 110, and a better sealing effect can be provided. Furthermore, the alignment boss 112 is located at each of two sides of the first feed-through plate 200, therefore the flexible neural electrode 300 can be subject to a more balanced force when the press cover 120 presses the flexible neural electrode 300, and an excellent electrical connection effect is ensured.

In some specific embodiments of the present disclosure, as shown in FIG. 4, a bottom surface of the press cover 120 is configured with a pressing plate 123 corresponding to a position of the first feed-through plate 200. The pressing plate 123 compresses the proximal contact portion 310 of the flexible neural electrode 300 against the interposer connector 400 in a value reaching a first threshold, to allow the proximal contact portion 310 of the flexible neural electrode 300 and the conductive contact of the first feed-through plate 200 of the implantable case 100 are electrically connected to each other through the interposer connector 400.

Furthermore, The bottom surface of the pressing plate 123 is flush with the bottom of the overall external outline of the press cover 120. The pressing plate 123 is configured to have a flat surface to which the flexible neural electrode 300 is attached. A shape of the pressing plate 123 is the same as a shape of the proximal contact portion 310. The pressing plate 123 presses the flexible neural electrode 300, so that the flexible neural electrode 300 experiences a more uniform force and the electrical connection position of the flexible neural electrode 300 with the interposer connector 400 is more accurate.

In some specific embodiments of the present disclosure, the first feed-through plate 200 includes a first insulating layer. The first conductive contact 210 passes through the first insulating layer along a thickness direction. Therefore, the first feed-through plate 200 has a number of conductive channels, and can maintain a smaller thickness. Furthermore, the first conductive contacts 210 are arranged on the first feed-through plate 200 in an array and the number thereof is not less than 100. Specifically, according to the requirements of signal transmission, the number of channels that can be readout in a parallel manner and the maximum number of connection lines that can be arranged in the probe rod can be used to connect the stimulation circuit or acquisition circuit.

Furthermore, the first insulating layer is made of ceramic or glass so as to have better insulation.

In some specific embodiments of the present disclosure, the proximal contact portion 310 of the flexible neural electrode 300 has a shape of polygon or circle. The interposer connector 400 has a shape matching the shape of the proximal contact portion 310 of the flexible neural electrode 300. A shape of the proximal contact portion 310 is adapted to a conductive area of the interposer connector 400. The interposer connector 400 is pressed against and is electrically connected to the proximal contact portion 310 through the first conductive contacts 210 on it, forms stimulation and acquisition surfaces in a rectangular or circular areas. The interposer connector 400 is connected to an electrode of the distal electrode site portion 330, so as to transmit an electrical signal of the neural signal circuit.

Furthermore, the flexible neural electrode 300 is made of a biocompatible material, which has high conductivity and chemical stability, so as to reduce rejection by the human tissue.

In some specific embodiments of the present disclosure, as shown in FIG. 2, the fully implantable and detachable high channel neural interface device 1 further includes a second feed-through plate 500. The second feed-through plate 500 is electrically connected to the proximal contact portion 310 of the flexible neural electrode 300. The second feed-through plate 500 is provided with a second conductive contact and is electrically connected to the distal electrode site portion 330 of the flexible neural electrode 300 via the interconnect portion 320 of the flexible neural electrode 300.

Furthermore, the second feed-through plate 500 includes a second insulating layer. The second conductive contact passes through the second insulating layer along a thickness direction. Furthermore, the second insulating layer is made of ceramic or glass so as to have good insulation.

The second feed-through plate 500 is connected between the proximal contact portion 310 of flexible neural electrode 300 and the interposer connector 400. The first feed-through plate 200 and the second feed-through plate 500 are arranged on opposite sides of the interposer connector 400. The second feed-through plate 500 is a rigid part. The second feed-through plate 500 is connected between the flexible neural electrode 300 and the interposer connector 400. The first feed-through plate 200 and the second feed-through plate 500 are connected on opposite sides of the interposer connector 400.

For example, the electrodes can be manufactured by micro-nano machining processes. Since the thickness of the electrode conductive contact obtained by micro-nano machining process is small, the second feed-through plate 500 can be configured and can be connected (e.g. welded) to the flexible neural electrode 300 in advance. Because the second feed-through plate 500 is a rigid part, deformation of the flexible neural electrode 300 can be overcome. The second feed-through plate 500 and the flexible neural electrode 300 form an integrated structure, which can effectively ensure the electrical connection reliability of the flexible neural electrode 300. Furthermore, the second feed-through plate 500 also has the function of positioning, which ensures that the flexible neural electrode 300 is connected to the first conductive contacts 210 arranged in an array in the first feed-through plate 200 via the second feed-through plate 500 and ensures electric connection accuracy.

In some specific embodiments of the present disclosure, the interposer connector 400 includes an insulating medium and a plurality of conductive connectors (not shown in the figure) arranged on the insulating medium. The proximal contact portion 310 of the flexible neural electrode 300 is electrically connected to the first conductive contacts 210 by the conductive connectors. Thus, the stability of the electrical connection between the flexible neural electrode 300 and the interposer connector 400 is ensured.

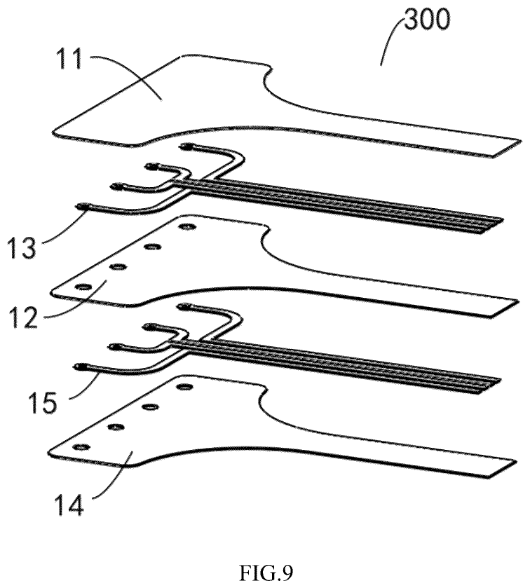

In some specific embodiments of the present disclosure, as shown in FIG. 9, the flexible neural electrode 300 is configured as a stacked structure and includes a first flexible insulating layer 11, a second flexible insulating layer 12 and a first conductive layer 13.

The first conductive layer 13 is positioned between the first flexible insulating layer 11 and the second flexible insulating layer 12. The first conductive layer 13 includes a plurality of electrodes. The plurality of electrodes each include a contact structure arranged at the proximal contact portion 310, an interconnecting wire arranged at the interconnect portion 320, and an electrode site structure arranged at the distal electrode site portion 330. The contact structure and the electrode site structure are exposed to at least one of the first flexible insulating layer 11 and the second flexible insulating layer 12.

Specifically, the flexible neural electrode 300 forms at least one layer of conductive structure. The plurality of electrodes of the first conductive layer 13 each extend from the distal electrode site portion 330 to the proximal contact portion 310. The contact structure and electrode sites are electrically connected to the interposer connector 400 and the first feed-through plate 200 by being exposed to at least one of the first flexible insulating layer 11 and the second flexible insulating layer 12. For example, a contact structure of of each of the plurality of electrodes, that is located at the proximal contact portion 310, corresponds to the position of the first conductive contact 210 of the first feed-through plate 200, so that the electric signal can be transmitted efficiently. The first flexible insulating layer 11 and the second flexible insulating layer 12 are insulated on opposite sides of the first conductive layer 13, which provides protection for electric connections.

Furthermore, the flexible neural electrode 300 further includes a third flexible insulating layer 14 and a second conductive layer 15. The second conductive layer 15 is positioned between the second flexible insulating layer 12 and the third flexible insulating layer 14. The second conductive layer 15 includes a plurality of electrodes. The plurality of electrodes each include a contact structure arranged at the proximal contact portion 310, an interconnecting wire arranged at the interconnect portion 320, and an electrode site structure arranged at the distal electrode site portion 330. The contact structure and the electrode site structure are exposed to at least one of the third flexible insulating layer 14, the second flexible insulating layer 12 and the first flexible insulating layer 11.

The flexible neural electrode 300 is configured with the first conductive layer 13 and the second conductive layer 15. The flexible neural electrode 300 is configured in a layered structure. Contact structures can be distributed in different positions of different layers of respective flexibilities. For example, the first conductive layer 13 and the second conductive layer 15 respectively correspond to the first conductive contacts at different positions of the first feed-through plate 200, so that the number of channel is increased and the rate of signal transmission is improved. The first flexible insulating layer 11, the second flexible insulating layer 12 and the third flexible insulating layer 14 are insulated on opposite sides of the first conductive layer 13 and the second conductive layer 15 respectively to provide protection for electrical connection.

In some specific embodiments of the present disclosure, the interposer connector 400 can be a conductive film. Each conductive film is electrically connected to the flexible neural electrode 300 and a neural signal circuit.

For example, the conductive film is a anisotropic conductive film, an LGA connector, or other two-dimensional lattice connectors with a first surface and a second surface, and is applied for the electric interconnection between the proximal contact portion 310 of the flexible neural electrode 300 for high-flux nerve and the device 1. Furthermore, the conductive film is a biocompatible material, which has high conductivity and chemical stability and thus can reduce rejection by the human tissue.

In some specific embodiments of the present disclosure, the device 1 further includes an external controller (not shown in the figure). The external controller is in communicative connection with the device 1 to control the operation of the device 1.

For example, the external controller communicates wirelessly with the device 1. The external controller controls the device 1 to generate pulses. An electrical stimulation to a nerve target is applied through the flexible neural electrode 300, so as to achieve a therapeutic effect. Furthermore, the external controller communicates with the acquisition circuit of the device 1, so as to brain neural signals to assist in the therapy.

In some specific embodiments of the present disclosure, the device 1 further includes a data processing module. The data processing module is configured to process an electric signal received from the flexible neural electrode 300 and/or generate an electric signal for controlling the flexible neural electrode 300.

The data processing module is connected to the neural signal acquisition circuit and/or the neural stimulation circuit. Through the neural signal acquisition circuit and the neural stimulation circuit, the data processing module collects electric signals from the surgical area and performs electric stimulation therapy.

In some specific embodiments of the present disclosure, the device 1 further includes a protection module. A battery module is arranged in the implantable case 100. When the fully implantable and detachable high channel neural interface device 1 is in malfunction or abnormal situation, the protection module performs shutoff protection. For example, the protection module includes an overcurrent protection, an overvoltage protection and an overheat protection. The protection module is configured to disconnect the neural signal acquisition circuit and the neural stimulation circuit when it is detected that the device 1 is abnormal, so as to prevent damage to human body caused by abnormal electrical stimulation.

In some specific embodiments of the present disclosure, the device 1 further includes an external controller. The external controller is equipped with a wireless communication module for wireless communication with the fully implantable and detachable high channel neural interface device 1.

The operating status of the device 1 can be controlled by the external controller. In this way, the volume of the device 1 implanted in the human brain can be reduced and the damage can be reduced. The external controller works with the device 1, to transmit wireless signals to the device 1, adjust the running status of device 1 in real time, so as to guarantee a better treatment result. In some embodiments, the external controller further includes a power module for providing electric power to the external controller.

In some specific embodiments of the present disclosure, the external controller is arrange with a display screen for displaying status information of the fully implantable and detachable high channel neural interface device. The display screen of the external controller can display information such as operating parameters and operating status of the device 1, so as to more visually show the therapeutic status and adjustment of the parameters and achieve an improved interaction. Furthermore, the external controller is arrange with buttons or a touch panel for inputting control instructions. By directly inputting parameters, it is possible to adjust the parameters more quickly and accurately.

Furthermore, the device 1 further includes an battery module. The battery module is arrange in the implantable case 100 for supplying electric power to the fully implantable and detachable high channel neural interface device 1. Furthermore, the battery module is a rechargeable battery. The external controller is equipped with a charging interface. The rechargeable battery is electrically connected to the battery module, and thus the rechargeable battery can be charged.

The device 1 is a detachable structure. The external controller is equipped with the rechargeable battery. The external controller is used to charge the battery module so as to keep the device 1 fully charged, and no additional charging device is required, and the structural integrity is stronger.

A method for producing a flexible neural electrode according to an embodiment of the present disclosure is described below.

The method for producing the electrode according to the embodiment of the present disclosure is applied to the flexible neural electrode in any of the above embodiments of the present disclosure. The method includes the following steps S1 to S5.

At step S1, a sacrificial layer is formed on a support by photolithography and metal coating.

At step S2, a first flexible insulating layer is formed on the sacrificial layer by spin coating.

At step S3, a first conductive layer is formed on the first flexible insulating layer by photolithography and metal deposition.

At step S4, a second flexible insulating layer is formed by spin coating on the first conductive layer and the first flexible insulating layer that have been formed;

At step S5, an overall structure of the flexible neural electrode is formed by etching the first flexible insulating layer and the second flexible insulating layer, and a contact structure of the proximal contact portion and an electrode site structure of the distal electrode site portion are exposed by etching the second flexible insulating layer.

At step 6, the flexible neural electrode is placed in a sacrificial layer removal solution to partially or completely detach the flexible neural electrode from the substrate, and the detached support is subsequently removed so as to obtain the freestanding flexible neural electrode.

The method according to the embodiment of the present disclosure, when applied to the flexible neural electrode in any of the embodiments of the present disclosure, can keep the electric electrode in human body, balance detachability and sealing performance, reduce the risk of operation and reduce the damage to the human body.

A method for replacing a fully implantable and detachable high channel neural interface device according to an embodiment of the present disclosure is described below.

The method for replacing the fully implantable and detachable high channel neural interface device according to the embodiment of the present disclosure can be applied to the device in any of the embodiments of the present disclosure. The method for replacing the fully implantable and detachable high channel neural interface device includes:
taking an original implantable case out;
implanting a new implantable case into a target tissue;
electrically connecting the proximal contact portion of an original flexible neural electrode to the first feed-through plate of the new implantable case.

Furthermore, when the original implantable case is taken out, the original neural electrode is not taken out.

Furthermore, when connecting the proximal contact portion of the flexible neural electrode to the first feed-through plate of the new implantable case, a detachable connection mode is adopted. The proximal contact portion of the flexible neural electrode is consistent with the contact of the new implantable case. The alignment pin can be matched to the proximal structure of the flexible neural electrode. In this way, electrodes can be connected directly to the new implantable case. When it is necessary to replace the implantable case, the press cover can be opened and thus it is very convenient to achieve separation of the flexible neural electrode from the implantable case.

The method for replacing the fully implantable and detachable high channel neural interface device according to the embodiment of the present disclosure, when applied to the device in any of the above embodiments, can keep the electrode in human body, balance detachability and sealing performance, reduce the risk of operation is small and reduce the damage to the human body.

The other components and operation of the fully implantable and detachable high channel neural interface device 1 according to the embodiment of the present disclosure are known to the person of ordinary skills in the art, and detailed description thereof will be omitted here.

In the description of the present specification, the description with reference to the terms “an embodiment”, “some embodiments”, “illustrative embodiment”, “example”, “specific example”, or “some examples”, etc. refer to particular features, structures, materials or characteristics described in the embodiments or examples being included in at least an embodiment or example of the present disclosure. In the present specification, the schematic representation of the above terms does not necessarily mean the same embodiment or example.

Although the embodiments of the present disclosure have been shown and described above, it can be appreciated by those skilled in the art that various changes, modifications, replacements and variants can be made to the above embodiments without departing from the principle and spirit of the present disclosure. The scope of the present disclosure is defined by the claims and their equivalents.

What is claimed is:

1-20. (canceled)

21. A fully implantable and detachable high channel neural interface device, comprising:
an implantable case comprising a bottom shell and a press cover, the press cover being removably mounted on the bottom shell and being sealed with the bottom shell, the implantable case being configured with a neural signal circuit therein;
a first feed-through plate arranged on the bottom shell, the first feed-through plate having a first surface facing an interior of the implantable case and a second surface facing away from the first surface, the first feed-through plate being provided with a plurality of first conductive contacts extending from the first surface to the second surface, the plurality of first conductive contacts being electrically connected to a neural signal circuit inside the implantable case at the first surface;
a flexible neural electrode comprising a proximal contact portion, an interconnect portion and a distal electrode site portion, the distal electrode site portion comprising a plurality of electrode sites and being electrically connected to the proximal contact portion by the interconnect portion, the proximal contact portion of the flexible neural electrode and an interposer connector being sealed between the press cover and the bottom shell, and the interconnect portion and the distal electrode site portion of the flexible neural electrode protruding out from between the bottom shell and the press cover, the proximal contact portion being a planar structure and being detachably connected to the bottom shell; and
the interposer connector arranged between the proximal contact portion of the flexible neural electrode and the second surface of the first feed-through plate, the proximal contact portion of the flexible neural electrode being electrically connected to the first conductive contact of the first feed-through plate via the interposer connector so as to allow the distal electrode site portion of the flexible neural electrode to be electrically connected to the neural signal circuit in the implantable case.

22. The fully implantable and detachable high channel neural interface device according to claim 21, wherein the interposer connector is configured in such a way that when a pressure applied by the press cover to the proximal contact portion of the flexible neural electrode reaches a first threshold, the proximal contact portion of the flexible neural electrode is electrically connected to the first conductive contact of the first feed-through plate via the interposer connector so as to allow the distal electrode site portion of the flexible neural electrode to be electrically connected to the neural signal circuit in the implantable case.

23. The fully implantable and detachable high channel neural interface device according to claim 21, wherein the neural signal circuit comprises a neural signal acquisition circuit and/or a neural signal stimulation circuit.

24. The fully implantable and detachable high channel neural interface device according to claim 21, wherein one side of the bottom shell is configured with a recess portion, the first feed-through plate, the interposer connector, and the proximal contact portion of the flexible neural electrode are all mounted on the recess portion, the press cover seals the first feed-through plate, the interposer connector, and the proximal contact portion of the flexible neural electrode to the recess portion.

25. The fully implantable and detachable high channel neural interface device according to claim 21, wherein a sealing member around the first feed-through plate is provided between the bottom shell and the press cover, the distal electrode site portion of the flexible neural electrode protrudes out of the implantable case by protruding out from between the sealing member and the press cover.

26. The fully implantable and detachable high channel neural interface device according to claim 21, wherein the bottom shell is configured with an alignment mechanism for positioning and mounting with the press cover, the interposer connector and the proximal contact portion of the flexible neural electrode.

27. The fully implantable and detachable high channel neural interface device according to claim 26, wherein the alignment mechanism comprises:
at least one alignment pin arranged on the bottom shell, the press cover, the interposer connector and the proximal contact portion of the flexible neural electrode all being configured with an alignment slot hole corresponding to a position of the alignment pin, the alignment pin passing through the alignment slot hole.

28. The fully implantable and detachable high channel neural interface device according to claim 27, wherein the alignment mechanism further comprises:
an alignment boss being arranged on the bottom shell and protruding towards a direction of the press cover; a bottom of the press cover being configured with a limiting groove that fits with the alignment boss.

29. The fully implantable and detachable high channel neural interface device according to claim 22, wherein a bottom surface of the press cover is configured with a pressing plate corresponding to a position of the first feed-through plate, the pressing plate compresses the proximal contact portion of the flexible neural electrode against the interposer connector in a value reaching the first threshold, to allow the proximal contact portion of the flexible neural electrode and the first conductive contact of the first feed-through plate of the implantable case are electrically connected to each other through the interposer connector.

30. The fully implantable and detachable high channel neural interface device according to claim 21, wherein the first feed-through plate comprises a first insulating layer, the first conductive contact passes through the first insulating layer along a thickness direction.

31. The fully implantable and detachable high channel neural interface device according to claim 21, further comprising:
a second feed-through plate, the second feed-through plate being electrically connected to the proximal contact portion of the flexible neural electrode, being provided with a second conductive contact, and being electrically connected to the distal electrode site portion of the flexible neural electrode via the interconnect portion of the flexible neural electrode.

32. The fully implantable and detachable high channel neural interface device according to claim 21, wherein the interposer connector comprises an insulating medium and a plurality of conductive connectors arranged on the insulating medium, and the proximal contact portion of the flexible neural electrode is electrically connected with the first conductive contact via the conductive connector.

33. The fully implantable and detachable high channel neural interface device according to claim 21, wherein the flexible neural electrode is configured as a stacked structure and comprises:
a first flexible insulating layer;
a second flexible insulating layer; and
a first conductive layer;
wherein the first conductive layer is positioned between the first flexible insulating layer and the second flexible insulating layer, the first conductive layer comprises a plurality of electrodes, the plurality of electrodes each comprising a contact structure arranged at the proximal contact portion, an interconnecting wire arranged at the interconnect portion, and an electrode site structure arranged at the distal electrode site portion;
the contact structure and the electrode site structure are exposed to at least one of the first flexible insulating layer and the second flexible insulating layer.

34. The fully implantable and detachable high channel neural interface device according to claim 33, wherein the flexible neural electrode further comprises:
a third flexible insulating layer; and
a second conductive layer;
wherein the second conductive layer is positioned between the second flexible insulating layer and the third flexible insulating layer, the second conductive layer comprises a plurality of electrodes comprising a contact structure arranged at the proximal contact portion, an interconnecting wire arranged at the interconnect portion, and an electrode site structure arranged at the distal electrode site portion;
the contact structure and the electrode site structure are exposed to at least one of the third flexible insulating layer and the second flexible insulating layer/the first flexible insulating layer.

35. The fully implantable and detachable high channel neural interface device according to claim 33, wherein the flexible neural electrode comprises: at least one of an epidural electroencephalogram electrode, a subdural electroencephalogram electrode, an intracortical electrode, and a depth electrode.

36. The fully implantable and detachable high channel neural interface device according to claim 21, further comprising: a data processing module configured to process a neural signal collected from the distal electrode site portion of the flexible neural electrode and/or generate a stimulation signal acting on the distal electrode site portion of the flexible neural electrode.

37. The fully implantable and detachable high channel neural interface device according to claim 21, further comprising a protection module configured to perform shutoff protection in case of malfunction or abnormal situation of the fully implantable and detachable high channel neural interface device.

38. The fully implantable and detachable high channel neural interface device according to claim 21, wherein the implantable case further comprises a battery module configured to provide electric power to the fully implantable and detachable high channel neural interface device.

39. The fully implantable and detachable high channel neural interface device according to claim 21, further comprising:
an external controller, the external controller being equipped with a wireless communication module for wireless communication with the fully implantable and detachable high channel neural interface device.

40. A method for producing a flexible neural electrode, being applied to the fully implantable and detachable high channel neural interface device according to claim 1 and comprising:
forming a sacrificial layer on a support by photolithography and metal coating (S1);
forming a first flexible insulating layer on the sacrificial layer by spin coating (S2);
forming a first conductive layer on the first flexible insulating layer by photolithography and metal deposition (S3);
forming a second flexible insulating layer by spin coating on the first conductive layer and the first flexible insulating layer that have been formed (S4);
forming an overall structure of the flexible neural electrode by etching the first flexible insulating layer and the second flexible insulating layer, and exposing a contact structure of the proximal contact portion and an electrode site structure of the distal electrode site portion by etching the second flexible insulating layer (S5); and
placing the flexible neural electrode in a sacrificial layer removal solution to partially or completely detach the flexible neural electrode from the substrate, and subsequently removing the detached support so as to obtain the freestanding flexible neural electrode (S6).
